(19) United States
(12) Patent Application Publication
(10) Pub. No.: US 2017/0360322 A1
(43) Pub. Date: Dec. 21, 2017

(54) MAGNETIC FIELD MEASURING APPARATUS AND CELL ARRAY

(71) Applicant: SEIKO EPSON CORPORATION, Tokyo (JP)

(72) Inventor: Hitoshi UENO, Shiojiri-shi (JP)

(73) Assignee: SEIKO EPSON CORPORATION, Tokyo (JP)

(21) Appl. No.: 15/614,963

(22) Filed: Jun. 6, 2017

(30) Foreign Application Priority Data
Jun. 15, 2016 (JP) 2016-118613

(51) Int. Cl. A61B 5/05; A61B 5/00; A61B 5/06; A61B 5/04

(52) U.S. Cl. CPC A61B 5/05; A61B 5/04007; A61B 5/0044; A61B 5/062; A61B 5/0042; A61B 5/0059; A61B 2562/0223; A61B 2562/046

(57) ABSTRACT

A magnetic field measuring apparatus includes a cell array including a plurality of cells each accommodating a medium that changes the polarization rotation angle of laser light incident on the medium in accordance with the intensity of a magnetic field, alight source that emits the laser light, and shielding members with which the cells are provided.

### BACKGROUND

### 1. Technical Field

The present invention relates to a magnetic field measuring apparatus and a cell array.

### 2. Related Art

There is a known optical-pumping-type magnetic sensor (magnetic field measuring apparatus) of related art that optically measures a weak magnetic field emitted from the heart or the brain. The optical-pumping-type magnetic sensor includes a cell in which a gas, such as an alkali metal, is encapsulated. An alkali metal atom is characterized in that the alkali metal atom irradiated with linearly polarized light changes the polarization rotation angle of the linearly polarized light in accordance with the magnitude of an applied magnetic field. The optical-pumping-type magnetic sensor is an apparatus that measures the intensity of the magnetic field by detecting the polarization rotation angle described above. In the field of the optical-pumping-type magnetic sensor, multi-channel measurement using a plurality of cells has been studied for expansion of the magnetic field measurement range and improvement in resolution of the magnetic field measurement.

For example, JP-A-2012-177585 proposes a magnetic field measuring apparatus in which partition walls are disposed between cells arranged in a matrix to prevent inter-cell optical crosstalk.

The magnetic field measuring apparatus described in JP-A-2012-177585, however, has a problem of a difficulty in improving easiness of maintenance of the cells and suppressing the inter-cell optical crosstalk at the same time. In detail, since the cells arranged in a matrix (cell array) are bonded to the partition walls and other components, for example, via low-melting glass, it is difficult to exchange a defective cell for a non-defective cell or remove the defective cell for repair. Similarly, also in the step of manufacturing the magnetic field measuring apparatus, it is difficult to discretely exchange a cell determined to be defective in finished product inspection for a non-defective cell. That is, a magnetic field measuring apparatus that allows suppression of optical crosstalk and improvement in easiness of maintenance of cells has been desired.

### SUMMARY

An advantage of some aspects of the invention is to solve at least a part of the problems described above, and the invention can be implemented as the following forms or application examples.

### Application Example

A magnetic field measuring apparatus according to this application example includes a cell array including a first cell and a second cell that each accommodate a medium that changes a polarization rotation angle of probing light incident on the medium in accordance with an intensity of a magnetic field, a light source that emits the probing light, and shielding members with which the first cell and the second cell are provided.

According to this application example, since the first cell and the second cell have the shielding members independent of each other, easiness of maintenance of the cells can be improved with inter-cell optical crosstalk suppressed. In detail, the shielding members are not a partition wall integrated with the cells, unlike the related art, but are provided independently of each other on a cell basis. Any of the cells can therefore be readily individually removed from the cell array including the first cell and the second cell. As a result, in maintenance of the magnetic field measuring apparatus and post-manufacture/inspection correction of the magnetic field measuring apparatus, easiness of the maintenance of the cells is improved. Further, since each of the first cell and the second cell is provided with the shielding member, optical shielding capability between the first cell and the second cell is improved. Noise originating from the inter-cell optical crosstalk can therefore be reduced. A magnetic field measuring apparatus having not only detection sensitivity improved by the suppression of the inter-cell optical crosstalk but also the improved cell maintenance can therefore be provided.

In the magnetic field measuring apparatus according to the application example described above, it is preferable that the shielding members are provided on adjacent surfaces of the first cell and the second cell.

According to the configuration described above, in the first cell and the second cell, light (fluorescence) outputted from one of the cells toward the adjacent cell and light (fluorescence) incident on one of the cells from the adjacent cell are both reduced. Therefore, in the magnetic field measurement, noise resulting from the inter-cell optical crosstalk is further reduced.

In the magnetic field measuring apparatus according to the application example described above, it is preferable that each of the first cell and the second cell has a first chamber on which the probing light is incident, and that the corresponding shielding member is provided on an outer shell that forms the first chamber but in an area excluding an area through which the probing light passes.

According to the configuration described above, the shielding members do not prevent transmission of the probing light for the magnetic field measurement. In addition thereto, the amount of fluorescence emitted from the first chamber of each of the cells and acting as noise in the magnetic field measurement is reduced.

In the magnetic field measuring apparatus according to the application example described above, it is preferable that the shielding members have a light absorbing property.

In the configuration described above, the shielding members having a light absorbing property can suppress scattering of fluorescence. The optical crosstalk in the magnetic field measuring apparatus can therefore be further suppressed.

In the magnetic field measuring apparatus according to the application example described above, it is preferable that the shielding members each include a fabric.

According to the configuration described above, the shielding members can be readily processed and placed. Further, the function of protecting the cells against impact or any other type of external force can be imparted.

In the magnetic field measuring apparatus according to the application example described above, it is preferable that the shielding members each include a resin layer.

According to the configuration described above, the shielding members (resin layers) can be made of a liquid material, which can be applied onto the cells. The shielding members can thus be placed on the cells. Therefore, even though the cells each have irregularities, the shielding members can each be seamlessly formed over a desired application range, whereby fluorescence shielding capability can be ensured.

In the magnetic field measuring apparatus according to the application example described above, it is preferable that the medium contains an alkali metal.

According to the configuration described above, the polarization plane orientation of the probing light emitted from the light source can be changed in accordance with the intensity of the magnetic field.

In the magnetic field measuring apparatus according to the application example described above, it is preferable that each of the first cell and the second cell accommodates a buffer gas.

According to the configuration described above, the buffer gas restricts movement of the medium in each of the cells and therefore prevents the medium from directly colliding with the inner wall of the cell, whereby the period for which an excited state produced by the radiated probing light attenuates can be prolonged. The excited state of the medium is therefore maintained for a longer period than in a case where no buffer gas is present, whereby the detection sensitivity of the magnetic field measuring apparatus can be improved.

In the magnetic field measuring apparatus according to the application example described above, it is preferable that a paraffin film containing aliphatic hydrocarbon having a carbon number of 20 or more is provided on an inner surface of each of the first cell and the second cell.

According to the configuration described above, the excited medium is unlikely to directly collide with the inner wall of each of the cells, whereby the period for which the excited state of the medium attenuates can be prolonged. The excited state of the medium is therefore maintained for a longer period than in a case where no paraffin film is provided, whereby a decrease in the detection sensitivity of the magnetic field measuring apparatus with time can be suppressed.

In the magnetic field measuring apparatus according to the application example described above, it is preferable that each of the first cell and the second cell has a first chamber on which the probing light is incident and a second chamber that communicates with the first chamber, and that each of the first cell and the second cell is provided with the shielding member.

According to the configuration described above, the amount of fluorescence produced in the first chamber and leaking through the second chamber is reduced. The optical crosstalk in the magnetic field measuring apparatus can therefore be further suppressed.

### Application Example

A cell array according to this application example includes at least a first cell and a second cell each accommodating a medium that changes a polarization plane orientation of probing light incident on the medium in accordance with an intensity of a magnetic field and shielding members with which the first cell and the second cell are provided, and the at least first cell and second cell are so disposed as to be adjacent to each other.

According to this application example, since the first cell and the second cell have the shielding members independent of each other, easiness of maintenance of the cells can be improved with inter-cell optical crosstalk suppressed.

### BRIEF DESCRIPTION OF THE DRAWINGS

The invention will be described with reference to the accompanying drawings, wherein like numbers reference like elements.

FIG. 1 is a block diagram showing the configuration of a magnetic field measuring apparatus according to a first embodiment.

FIG. 2 is a schematic plan view showing the configuration of a cell array.

FIG. 3 is a schematic cross-sectional view showing the configuration of the cell array.

FIG. 4 is a schematic perspective view showing a cell.

FIG. 5 is a schematic perspective view showing a primary chamber and a secondary chamber of a cell according to a second embodiment.

FIG. 6 is a schematic perspective view showing a cell according to variation 1.

FIG. 7 is a schematic perspective view showing a cell according to variation 2.

### DESCRIPTION OF EXEMPLARY EMBODIMENTS

Embodiments of the invention will be described below with reference to the drawings. In the following figures, each part and each member are so drawn at scales different from actual scales as to be large enough to be recognizable.

### First Embodiment

### Magnetic Field Measuring Apparatus

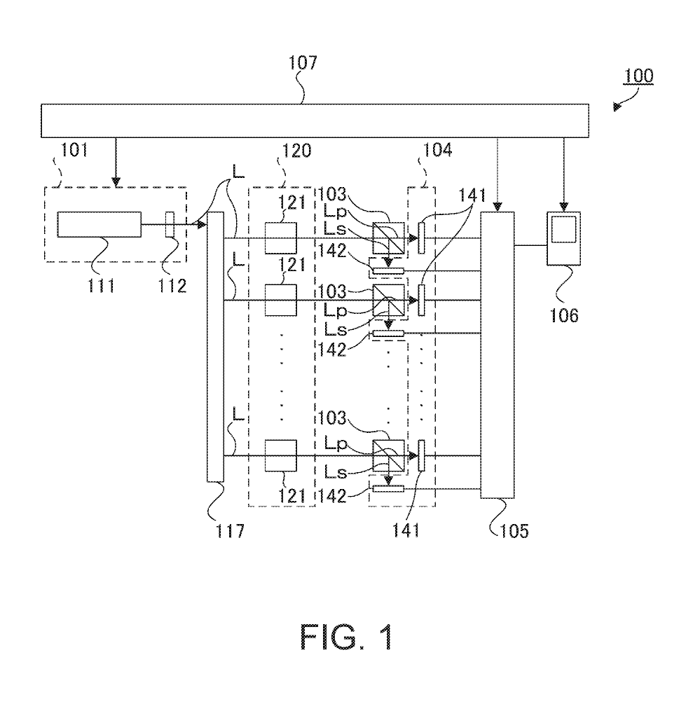

The configuration of a magnetic field measuring apparatus according to an embodiment of the invention will be described with reference to FIG. 1. FIG. 1 is a block diagram showing the configuration of a magnetic field measuring apparatus according to a first embodiment.

The present embodiment will be described with reference to what is called a one-beam magnetic field measuring apparatus that measures a magnetic field by using nonlinear magneto-optical rotation (NMOR). In the one-beam magnetic field measuring apparatus, a cell is irradiated with laser light containing linearly polarized light to achieve a state in which medium (alkali metal) atoms accommodated in the cell are excited. The laser light having passed through the cell is then detected for magnetic field measurement. That is, the one beam method is a method in which one beam is used not only as pumping light for exciting the medium atoms but also as probing light (linearly polarized light) for detecting the polarization rotation angle changed by the excited medium atoms.

A magnetic field measuring apparatus 100 shown in FIG. 1 includes a light radiator 101 and a cell array 120 including a plurality of cells 121. The magnetic field measuring apparatus 100 further includes a splitting optical element 117, polarization separation elements 103, a light receiver 104, a signal processor 105, a display section 106, and a controller 107.

The light radiator 101 includes a light source 111 and a converter 112. The light source 111 is a laser light generator that outputs laser light L containing linearly polarized light as the probing light and is, for example, a tunable laser. The laser light L is what is called CW (continuous wave) light, which is continuously radiated and has a fixed amount of light. The output of the light source 111 is so adjusted that the amount of laser light L incident on the cells 121 is about several tens of microwatts. The converter 112 is, for example, a polarizer and converts the laser light L emitted by the light source 111 into linearly polarized light having a polarization angle in a predetermined direction with respect to the optical axis.

The single laser light L outputted from the light radiator 101 is incident on the splitting optical element 117. The splitting optical element 117 splits the laser light L incident thereon to cause the laser light L to be incident on each of the cells 121.

Each of the cells 121 accommodates a medium that changes the polarization plane orientation of the laser light L incident on the cell in accordance with the intensity of a magnetic field. The medium is preferably an alkali metal, which can be vaporized at a relatively low temperature, specifically, potassium (K) and cesium (Cs). The alkali metal accommodated in each of the cells 121 is at least partially vaporized at the time of magnetic field measurement. In the present embodiment, cesium is used as the medium. The laser light L described above is therefore so adjusted as to have the wavelength according to an absorption line of cesium (894 nm corresponding to D1 line, for example).

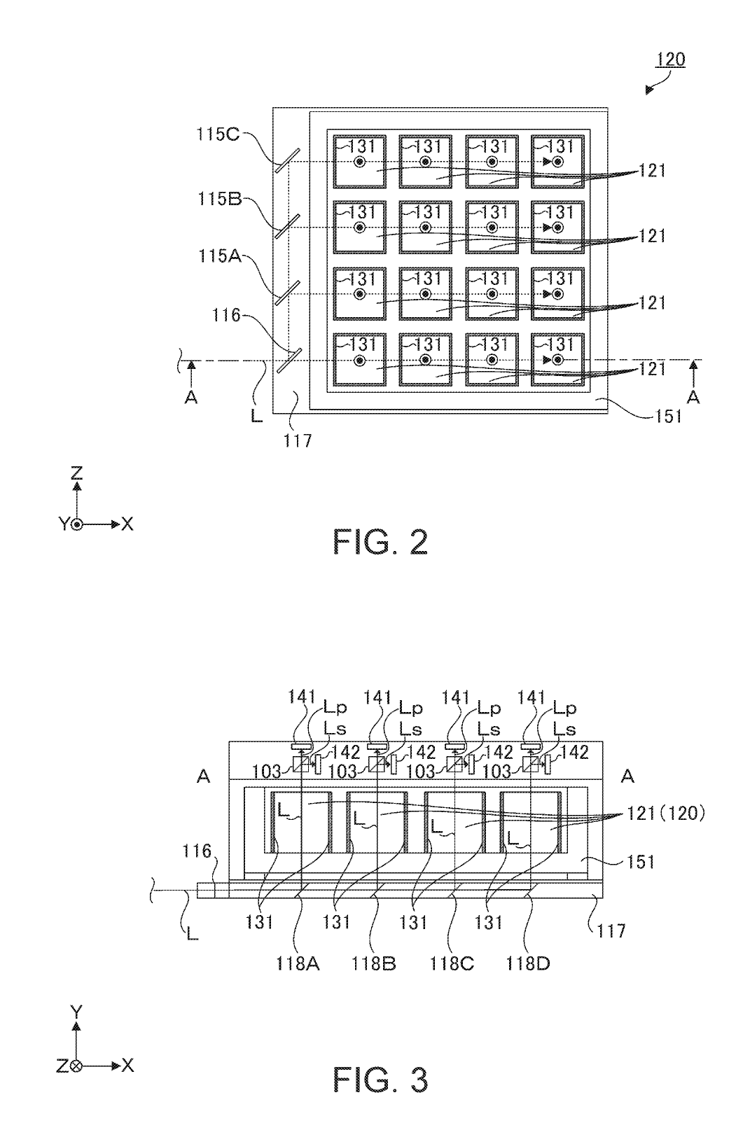

An outer shell of each of the cells 121 is made, for example, of quartz, which transmits the laser light L. The outer shell of each of the cells 121 can be made of any material that can transmit the laser light L but does not react with an alkali metal or any other medium, and an organic material can also be used in place of an inorganic material, such as quartz and borosilicate glass. The plurality of cells 121 are so arranged as to be adjacent to each other to form the cell array 120. Use of the cell array 120 including a plurality of cells 121 allows expansion of the magnetic field measurement range and improvement in measurement resolution. Each of the plurality of cells 121 is provided with a spieling member 131 (see FIG. 2), which will be described later. Among the plurality of cells 121, one of cells 121 adjacent to each other corresponds to the first cell according to an aspect of the invention, and the other corresponds to the second cell according to the aspect of the invention.

The laser light L attenuates when it is reflected off the surface of the outer shell of each of the cells 121 and absorbed by the interior of the cell 121. The alkali metal atoms as the medium described above, which absorb the linearly polarized light contained in the laser light L, repeats transition between the ground state and the excited state to form a specific energy distribution (spin polarization; alignment). When a magnetic field is applied to the cell in a state in which the energy distribution (spin polarization; alignment) is maintained, the atoms described above anisotropically absorbs the linearly polarized light. That is, the state of the spin polarization (alignment) changes. The linearly polarized light incident on each of the cell 121 is affected by the change in the spin polarization (alignment) so that the polarization plane orientation (polarization rotation angle) changes. As a result, the laser light L having the changed polarization plane orientation (polarization rotation angle) exits from the cell 121 and is incident on the corresponding polarization separation element 103.

The polarization separation elements 103 are disposed in correspondence with the plurality of cells 121. The polarization separation elements 103 transmit a linearly polarized light component (P-polarized light component) having the same polarization direction (polarization plane orientation) as that of the linearly polarized light component of the laser light L converted by the converter 112 but reflect a linearly polarized light component (S-polarized light component) having a polarization direction perpendicular to the polarization direction of the linearly polarized light component of the laser light L. Each of the polarization separation elements 103 can, for example, be a polarizing beam splitter or a Wollaston prism. In the polarization separation elements 103, the light passing therethrough is called polarized light Lp, and the light reflected therefrom is called polarized light Ls. The polarized light Lp and the polarized light Ls are incident on the light receiver 104.

The light receiver 104 includes light receiving devices 141 and 142. One polarization separation element 103 is provided with one light receiving device 141 and one light receiving device 142. The light receiving devices 141 and 142 are each a detector sensitive to the wavelength of the laser light L. The light receiving devices 141 are each disposed in a position where it can receive the polarized light Lp, and the light receiving devices 142 are each disposed in a position where it can receive the polarized light Ls. The light receiving devices 141 each output current (signal) according to the amount of received polarized light Lp and transmit the signal to the signal processor 105. The light receiving devices 142 each output current (signal) according to the amount of received polarized light Ls and transmit the signal to the signal processor 105. The light receiving devices 141 and 142 are preferably made of a nonmagnetic material that does not interfere with the measurement performed by the magnetic field measuring apparatus 100. In the present specification, the “nonmagnetic” material means a material having no magnetism.

The signal processor 105 receives the signals described above and transmitted from the light receiving devices 141 and 142. The signal processor 105 measures, from the signals described above, the amount of change in the polarization rotation angle having changed when the linearly polarized light contained in the laser light L passes through the cells 121, that is, the angle of rotation of the polarization plane orientation.

The polarization separation elements 103, the light receiver 104, and the signal processor 105 have the function of detecting the angle of rotation of the polarization plane orientation having changed in the cells 121. Since the angle of rotation of the polarization plane orientation changes in accordance with the magnitude (intensity) of the magnetic field applied to the cells 121, the magnetic field measuring apparatus 100 can measure the magnitude of the magnetic field applied in a predetermined direction (measurement direction) to the cells 121 by detecting the angle of rotation of the polarization plane orientation described above.

The display section 106 is electrically connected to the signal processor 105. The display section 106 displays, for example, the angle of rotation described above measured by the signal processor 105. The display section 106 is, for example, a display apparatus using a liquid crystal panel or any other component.

The controller 107 includes a processing device (not shown), such as a CPU (central processing unit), and a memory (not shown). The controller 107 is electrically connected to the light radiator 101, the signal processor 105, the display section 106, and other components and has the function of synthetically controlling the actions thereof.

### Cell Array

The configurations of the cell array 120 and the splitting optical element 117, which is associated with the cell array 120, will next be described with reference to FIG. 2. FIG. 2 is a schematic plan view showing the configuration of the cell array. In FIG. 2, the laser light L is drawn with a dotted line.

The cell array 120 shown in FIG. 2 includes the plurality of cells 121, each of which accommodates a medium that changes the polarization plane orientation of the laser light L incident on the cell in accordance with the intensity of the magnetic field, and the shielding members 131, with which the cells 121 are provided. In the cell array 120, the plurality of cells 121 are arranged in the form of a matrix in a cell container 151. The cell array 120 is provided with the splitting optical element 117 via the cell container 151.

The cell container 151 is made of a material that transmits the laser light L. The material of which the cell container 151 is made can, for example, be quartz. In FIG. 2, directions are defined as follows: The direction roughly parallel to the longitudinal arrangement of the cells 121 is called a Z direction (upward direction is called positive direction); the direction roughly parallel to the lateral arrangement of the cells 121 and perpendicular to the z direction is called an X direction (rightward direction is called positive direction); and the direction perpendicular to the Z direction and the X direction is called a Y direction. The traveling direction of the laser light L passing through the cell array 120 is roughly parallel to the Y direction.

The cell array 120 includes 16 cells 121 in total that form 4 rows and columns, each of which is formed of 4 cells 121, in the X and Z directions, as shown in FIG. 2. Adjacent cells 121 are so disposed as to be separate from each other by a predetermined distance. Any of the cells 121 can therefore be individually removed from the magnetic field measuring apparatus 100, and maintenance, such as exchange and repair, can be readily performed on the removed cell 121. The predetermined distance described above ranges, for example, from about 0.1 to 10 mm.

The cells 121 have a roughly cubic shape and include the shielding members 131. Each of the shielding members 131 is so provided as to be in contact with the surfaces of the corresponding cell 121 that are roughly parallel to the Y direction (surfaces excluding surfaces roughly perpendicular to Y direction). The surfaces of each of the cells 121 other than the surface on which the laser light L is incident or the surface through which the laser light L exits (surfaces roughly parallel to Y direction) are hereinafter also referred to as “side surfaces.”

The splitting optical element 117 has the function of splitting the laser light L incident thereon to cause the laser light L to be incident on each of the cells 121, as described above. The splitting optical element 117 includes mirrors 115A to 115C, 116, and 118A to 118D (see FIG. 3). The laser light L incident on the splitting optical element 117 travels in the X direction (positive direction thereof) and first reaches the mirror 116. The mirror 116 reflects part of the laser light L in the Z direction (positive direction thereof) and transmits the remainder.

The mirrors 115A, 115B, and 115C are provided along the traveling direction of the laser light L reflected off the mirror 116 (Z direction). The three mirrors 115A, 115B, and 115C are disposed in the positions corresponding to the three rows formed of cells 121 and arranged in the Z direction. The laser light L reflected off the mirror 116 first reaches the mirror 115A. The mirror 115A reflects part of the laser light L in the X direction (positive direction thereof) and transmits the remaining laser light L. Similarly, the mirrors 115B and 115C reflect or transmit the laser light L to cause the laser light L to travel in the X direction (positive direction thereof) in correspondence with the rows of cells 121, which are roughly parallel to the X direction. The mirror 115C may be assumed to totally reflect the laser light L.

The configurations of the cell array 120 and the splitting optical element 117, the polarization separation elements 103, and other components associated with the cell array 120 will next be described with reference to FIG. 3. FIG. 3 is a schematic cross-sectional view showing the configuration of the cell array. FIG. 3 is a cross-sectional view of the cell array 120 taken along the line A-A shown in FIG. 2 and shows the splitting optical element 117, the polarization separation elements 103, and other components. It is assumed that the upward direction of the Y direction in FIG. 3 is called a positive direction.

The laser light L having passed through the mirror 116 travels in the X direction (positive direction thereof). The mirrors 118A to 118D are provided in the positions corresponding to the four columns formed of cells 121 and arranged in the X direction. The laser light L having passed through the mirror 116 first reaches the mirror 118A. The mirror 118A reflects part of the laser light L in the Y direction (positive direction thereof) to cause the laser light L to be incident on the corresponding cell 121. The mirror 118A transmits the remaining laser light L to cause the laser light L to reach the mirror 118B. Similarly, the mirrors 118B, 118C, and 118D reflect or transmit the laser light L to cause the laser light L to be incident on the corresponding cells 121. The mirror 118D may be assumed to totally reflect the laser light L.

In the cell array 120, the configurations in the X direction excluding the configuration in the A-A cross section are the same as the configuration in the A-A cross section described above except that the mirror 116 is replaced with the mirrors 115A to 115C. The laser light L reflected off the mirrors 115A to 115C in the X direction (positive direction thereof) is therefore radiated to the corresponding cells 121 via the mirrors 118A, 118B, 118C, and 118D arranged in the X direction, as in the case of the laser light L having passed through the mirror 116 described above.

Each of the mirrors 116, 115A to 115C, and 118A to 118D can, for example, be a partial polarizing beam splitter or a non-polarizing beam splitter having constant transmittance irrespective of the polarization plane orientation. It is preferable that the same amount of laser light L is incident on the 16 cells 121. To this end, the transmittance and reflectance of the mirrors 116, 115A to 115C, and 118A to 118D at which the mirrors transmit and reflect the linearly polarized light contained in the laser light L are so adjusted that the same amount of light described above is achieved.

In the configuration described above, the single laser light L is split by the splitting optical element 117, and each of the 16 cells 121 is irradiated with one corresponding laser light L.

Each of the shielding members 131 is provided on the side surfaces (surfaces roughly parallel to Y direction) of the corresponding cell 121. Since each of the shielding members 131, which will be described later, is a light absorbent member that absorbs light, such as the laser light L, optical crosstalk between the cells 121 is suppressed.

The laser light L passes through the cells 121, where the polarization plane orientation of the laser light L changes in accordance with the intensity of the magnetic field, as described above. The laser light L having passed through the cells 121 is separated by the polarization separation elements 103 into the polarized light Lp and the polarized light Ls, which are received with the light receiving devices 141 and 142, respectively. As described above, the laser light L radiated from the light radiator 101 passes through the cells 121, is then received with the light receiving devices 141 and 142, and undergoes the magnetic field measurement.

In FIG. 3, the magnetic field measuring apparatus 100 has been described as an apparatus based on what is called a single-pass method, in which the laser light L passes through the cells 121 only once, but the method is not necessarily employed. For example, the magnetic field measuring apparatus 100 may be an apparatus based on a multi-pass method, in which the laser light L passes through the cells 121 multiple times via mirrors and other components. Further, the laser light L does not necessarily pass through the cells 121 roughly in parallel to the Y direction.

### Cell

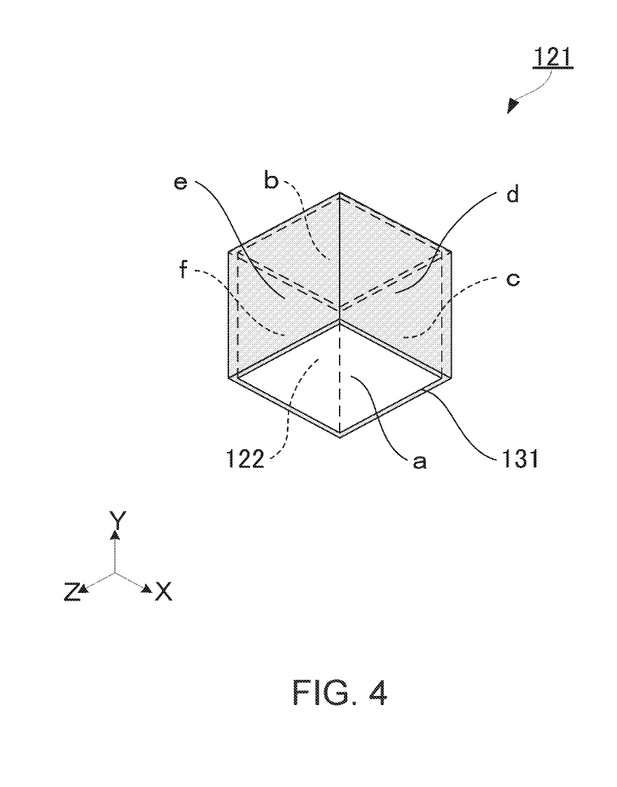

The configuration of the cells in the present embodiment will be described with reference to FIG. 4. FIG. 4 is a schematic perspective view showing a cell.

The cell 121 shown in FIG. 4 has a primary chamber 122, which is an example of a first chamber, and the shielding member 131 in the present embodiment. The primary chamber 122 is an internal space surrounded by a roughly cubic outer shell. The length of one side of the outer shell described above is, for example, about 2 cm. The laser light L enters the primary chamber 122. The outer shell described above is formed of 6 surfaces, surfaces a, b, c, d, e, and f each having a roughly square shape. The primary chamber 122 at least partially encapsulates gaseous cesium as the alkali metal (medium), and the outer shell maintains airtightness of the cell.

The surface a is the surface on which the laser light L is incident, and the surface b is the surface through which the laser light L having passed through the cell 121 exits. The surfaces c, d, e, and f are surfaces (side surfaces) of the outer shell that are roughly parallel to the Y direction and include surfaces via each of which two cells 121 are adjacent to each other when the entire cells 121 are arranged to form the cell array 120.

When the laser light L is incident on the cells 121, the alkali metal atoms in the cells 121 (primary chambers 122) are brought into the excited state, as described above. When the excited state returns (transitions) to the ground state, fluorescence is emitted. The fluorescence that accompanies the transition is so produced that the type of polarization thereof is determined by the energy levels in an eigenstate that causes the transition. In the case of the transition to the ground state described above, since the transition occurs at the same probability for all energy levels in the eigenstate, the emitted fluorescence contains linearly polarized light and circularly polarized light mixed with each other.

The fluorescence described above could enter adjacent cells 121, resulting in optical crosstalk. In particular, in measurement of a weak magnetic field in biological tissue, such as the heart and the brain, the detection sensitivity (resolution) decreases in some cases due to noise originating from the optical crosstalk. To suppress the emission and entry of the fluorescence described above, the surfaces c, d, e, and f may be provided with the shielding member 131. That is, the surfaces a and b are provided with no shielding member 131. The shielding member 131 is so placed as to seamlessly cover the surfaces c, d, e, and f. Further, the shielding member 131 is preferably provided in the area excluding the area through which the laser light L passes. That is, covering the outer shell of each of the cells 121 over a wide range thereof to the extent that the shielding member 131 does not block the transmission of the laser light L allows further suppression of the optical crosstalk between the cells 121. Therefore, in addition to the side surfaces of each of the cells 121, the surface a, on which the laser light L is incident, and the surface b, through which the laser light L exits, may be provided with the shielding member 131 except the area through which the laser light L passes. As a result, the optical shielding capability of the cells 121 is further improved.

The shielding members 131 are each a light absorbent member. That is, each of the shielding members 131 is preferably a light absorbent member that appropriately absorbs the fluorescence from cells 121 adjacent to the shielding member 131. For example, in a case where the alkali metal atoms of the medium are cesium atoms and the D1 absorption line is used, the transmittance of the shielding members 131 at which they transmit light having wavelengths close to the wavelength of 894 nm is preferably set to be lower than or equal to 0.01%. When the shielding members 131 are each the light absorbent member described above, a situation in which the fluorescence described above incident on the shielding members 131 is emitted as transmitted light or scattered light is avoided. The optical shielding capability of the cells 121 is therefore be improved.

Further, the shielding members 131 are each preferably a nonmagnetic member. When the shielding members 131 are each a nonmagnetic member, influence of a magnetic field originating from the shielding members 131 on the alkali metal accommodated in the cells 121 decreases. The detection sensitivity of the magnetic field measuring apparatus 100 can therefore be improved.

The shielding members 131 each preferably contain a fabric. The material of which the fabric is made is not limited to a specific material and may, for example, be cotton, hemp, and other vegetable fibers, silk, wool, and other animal fibers, polyester, acetate, polyamide, and other synthetic fibers, polylactic acid and other biodegradable fibers. The shielding members 131 can be formed by processing at least one of the materials described above into a fabric, such as a woven fabric, a knitted fabric, and a nonwoven fabric. The fabrics described above are preferably colored, for example, black by a coloring agent for an increase in the light absorbing capability of the fabrics. The shielding members 131 in the present embodiment are formed of a fabric produced by coloring a polyester nonwoven fabric black by using a coloring agent primarily made of carbon black.

In the case where a fabric is used as each of the shielding members 131, a method for placing the fabric on each of the cells 121 can, for example, be a method in which the cell 121 is covered with the fabric by using the elasticity thereof or a method in which an adhesive layer is provided between the outer shell of the cell 121 and the fabric. The thickness of the fabric used as each of the shielding members 131 is not limited to a specific value and may be any value that ensures the light absorbing capability. For example, the thickness can range from about 0.1 to 0.5 mm. The shielding members 131 are not necessarily in contact with the outer shells of the cells 121.

Instead of using a fabric, the shielding members 131 each preferably include a resin layer. The material of which the resin layer is made is not limited to a specific material and can, for example, be an acrylic resin, a urethane-based resin, a polyolefin-based resin, a polyester-based resin, a polyamide-based resin, an epoxy-based resin, and a vinyl-chloride-based resin. At least one of the resins described above can be used as the material of the resin layer. A coloring agent, such as a black coloring agent, is preferably added to the resin layer to increase the light absorbing capability of the resin layer.

In the case where the resin layer is used as each of the shielding members 131, a method for placing the resin layer on each of the cells 121 can be a method in which a liquid raw material of the resin layer is applied onto the outer shell of the cell 121 followed by solidification of the liquid raw material. Instead, a resin layer formed in the shape of a sheet in advance may be placed on the outer shell. The thickness of the resin layer used as each of the shielding members 131 is not limited to a specific value and may be any value that ensures the light absorbing capability. For example, the thickness can range from about 0.05 to 0.1 mm.

Further, the shielding members 131 may each be formed of the combination of the fabric and the resin layer described above. To improve the light absorbing capability, both the fabric and the resin layer may be superimposed on each other and placed as the shielding members 131 on the cells 121 (outer shells). Instead, the fabric and the resin layer may be properly used in accordance with the portion of the outer shell on which the shielding member 131 is placed. The cells 121 arranged along the outer edge of the cell array 120 may be so configured that no shielding member 131 is placed on the side surfaces of the cells 121 except the surfaces adjacent to each other.

A paraffin film (not shown) containing aliphatic hydrocarbon having a carbon number of 20 or more may be provided on the inner surface of each of the cells 12 (primary chambers 122). The paraffin film containing paraffin in the form of aliphatic hydrocarbon having a carbon number of 20 or more causes the atoms in the excited medium (alkali metal) to be unlikely to directly collide with the inner wall (inner surface) of the cell, whereby the period for which the excited state of the medium attenuates can be prolonged. The excited state of the medium is therefore maintained for a longer period than in a case where no paraffin film is provided, whereby a decrease in the detection sensitivity of the magnetic field measuring apparatus 100 with elapsed time can be suppressed.

The cells 121 (primary chambers 122) may accommodate an inert gas, such as a rare gas, as a buffer gas. When each of the cells 121 accommodates the buffer gas, the buffer gas restricts movement of the medium in the cell 121, preventing the medium from directly colliding with the inner wall of the cell. As a result, the period for which the excited state produced by the radiated laser light L attenuates can be prolonged. The excited state of the medium is therefore maintained for a longer period than in a case where no buffer gas is present, whereby a decrease in the detection sensitivity of the magnetic field measuring apparatus 100 with elapsed time can be suppressed.

As described above, the magnetic field measuring apparatus 100 and the cell array 120 according to the embodiment described above can provides the following effects.

According to the embodiment described above, since the plurality of cells 121 include the shielding members 131 independent of one another, easiness of maintenance of the cells 121 can be improved with optical crosstalk between the cells 121 suppressed. In detail, since the optical shielding capability of the cells 121 is improved, optical crosstalk between adjacent cells 121 can be suppressed. Further, since the cells 121 are independently provided with respective shielding members 131, any of the cells 121 can be readily individually removed from the cell array 120 and exchanged to a new cell. As a result, the maintenance of the cells 121 can be performed more readily than in related art. Therefore, in the magnetic field measuring apparatus 100 and the cell array 120 provided by the embodiment, the detection sensitivity improved by suppression of optical crosstalk, and easiness of the maintenance of the cells 121 are improved.

At least the adjacent surfaces of the cells 121 are provided with the shielding members 131, which reduce the amount of fluorescence emitted through the side surfaces of the cells 121. Therefore, both the fluorescence emitted from a cell 121 toward the adjacent cells 121 and the fluorescence incident on a cell 121 from the adjacent cells 121 can be reduced. As a result, optical crosstalk between the cells 121 can be suppressed.

Use of the fabric (polyester nonwoven fabric) colored black as each of the shielding members 131 further improves the capability of absorbing the fluorescence and further suppresses the optical crosstalk. Further, when the shielding members 131 each include the fabric, the function of protecting the cells against impact (external force) acting when the apparatus is handled can be imparted in addition to the easiness of processing and placement of the shielding members 131. Moreover, use of a nonmagnetic fabric allows improvement in the detection sensitivity of the magnetic field measuring apparatus 100.

### Second Embodiment

### Cell

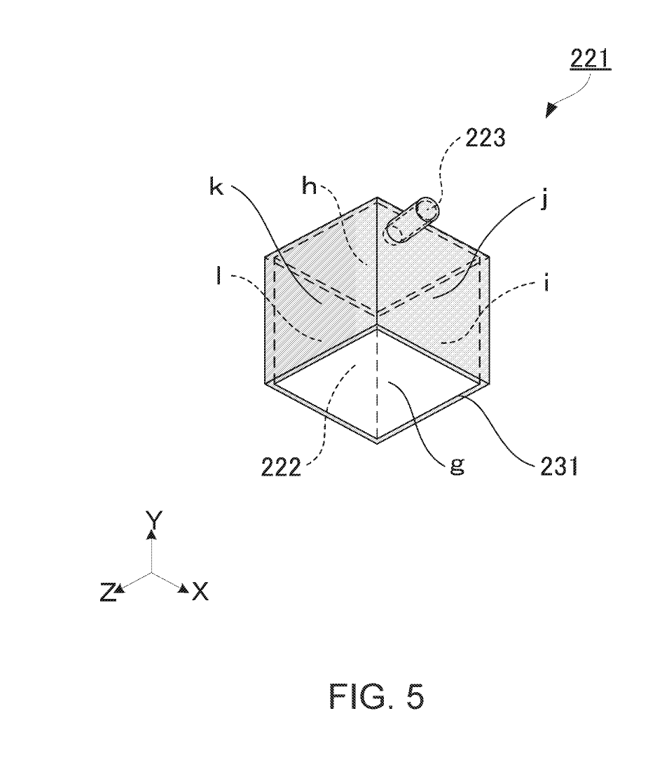

The configuration of cells according to the present embodiment will be described with reference to FIG. 5. FIG. 5 is a schematic perspective view showing a primary chamber that is an example of the first chamber and a secondary chamber that is an example of a second chamber in a cell according to the second embodiment. The same configuration portions as those in the first embodiment have the same reference characters, and no redundant description will be made.

A cell 221 shown in FIG. 5 has a primary chamber 222, which serves as the first chamber on which the laser light L is incident, and a secondary chamber 223, which serves as the second chamber that communicates with the primary chamber 222. The internal space surrounded by outer shells of the primary chamber 222 and the secondary chamber 223 encapsulates cesium as the medium, as in the first embodiment. The outer shells of the primary chamber 222 and the secondary chamber 223 are each provided with a shielding member 231.

The outer shell of the primary chamber 222 is formed of 6 surfaces, surfaces g, h, i, j, k, and l (alphabet) each having a roughly square shape. The secondary chamber 223 has a roughly cylindrical shape, is in contact with an outer edge portion of the surface j, and protrudes from the outer shell of the primary chamber 222. The outer shells of the primary chamber 222 and the secondary chamber 223 maintain airtightness of the interior of the cell 221. The outer shells of the primary chamber 222 and the secondary chamber 223 can be made of the same material as that in the first embodiment. In the present embodiment, the material of which the outer shells of the primary chamber 222 and the secondary chamber 223 are made is quartz.

Among the surfaces g, h, i, j, k, and l, the surface g is the surface on which the laser light L is incident, and the surface h is the surface through which the laser light L having passed through the cell 221 exits. The surfaces i, j, k, and l are surfaces (side surfaces) of the outer shell of the primary chamber 222 that are roughly parallel to the Y direction and include surfaces via each of which two cells 221 are adjacent to each other when the entire cells 221 are arranged to form a cell array 220 (not shown).

The secondary chamber 223 is provided as an introduction portion for introducing cesium (medium) or any other substance into the cell 221 (primary chamber 222) in the step of manufacturing the cell 221. After cesium or any other substance is introduced into the primary chamber 222 via an opening provided in the outer shell of the secondary chamber 223, the opening described above is closed, for example, with a heated sealing material. According to the method described above, the thermal burden imposed on the accommodated cesium or any other substance when the opening is closed can be reduced, as compared with the case where the opening is provided in the primary chamber 222.

Further, providing the secondary chamber 223 allows employment of a manufacturing method in which an ampule containing cesium, a buffer gas, or any other substance is introduced via the opening described above into the secondary chamber 223. In the manufacturing method, after the ampule described above is introduced into the secondary chamber 223, the opening described above is closed. Thereafter, the ampule is so irradiated, for example, with laser light as to be unsealed, and the cesium or any other substance can transpire across the primary chamber 222 and can be accommodated therein. The manufacturing method also allows reduction in the thermal burden imposed on the cesium or any other substance.

The shielding member 231 is so provided as to cover the entire outer shell of the secondary chamber 223 and the surfaces i, j, k, and l of the primary chamber 222. That is, the surfaces g and h are provided with no shielding member 231. The shielding member 231 can suppress the fluorescence emitted from the cell 221 and the fluorescence incident on the cell 221. The shielding member 231 includes a resin layer that is a light absorbent, nonmagnetic layer. The resin layer can be made of any of the materials described in the first embodiment. In the present embodiment, the shielding member 231 is made of an acrylic material, and a coloring agent primarily made of carbon black is added to the acrylic material for improvement in the light absorbent capability.

A method for placing the resin layer can be any of the methods described above. In the present embodiment, an emulsion which is formed of a medium primarily made of water and in which fine particles made of the acrylic resin and the carbon black pigment described above are dispersed is used as the resin layer. The emulsion described above, which is a liquid, is applied onto the cell 221, and the shielding member 231 can thus be placed. The thus applied emulsion described above is then dried to forma coating (resin layer). According to the method described above, the shielding member 231 can be readily placed also on the outer shell of the secondary chamber 223, which protrudes from the cell 221. That is, the method is suitable for placement of the shielding member 231 on an irregular area where it is difficult to place a fabric shielding member.

In the present embodiment, the aqueous acrylic resin emulsion is used to place the shielding member 231, but not necessarily used. For example, a non-aqueous emulsion or a heat curable or energy ray curable coating material may be used to place the shielding member 231. Still instead, a method in which a resin layer is formed in a sheet-like shape in advance and then the resin layer is placed on the cell 221 may be used.

In the present embodiment, the secondary chamber 223 is provided on a side surface (surface j) of the cell 221, but not necessarily limited thereto. For example, the secondary chamber 223 may be provided on the surface g or h, through which the laser light L passes but in an area other than the area through which the laser light L passes. Further, the shape of the secondary chamber 223 is not limited to a roughly cylindrical shape. The secondary chamber 223 may have any other shape, such as a columnar shape or a pyramidal shape, or the cross-sectional shape of part of the secondary chamber 223 may differ from the cross-sectional shape of the remainder.

As described above, the second embodiment differs from the first embodiment in that the cells 221 each have the secondary chamber 223, which is in contact with the primary chamber 222, and that the outer shell of the secondary chamber 223 is also provided with the shielding member 231. As described above, the cells 221 according to the embodiment described above can provide the following effects in addition to those provided by the first embodiment.

According to the embodiment described above, since the shielding member 231 includes the resin layer, the shielding member 231 can be made of a liquid material. The shielding member 231 can therefore be placed in an application process. The shielding member 231 can therefore be placed even on a placement area, for example, having irregularities, whereby the shielding capability of the cells 221 can be improved. Further, in the cell array 120 and the magnetic field measuring apparatus including the cells 221, optical crosstalk is suppressed and detection sensitivity can be improved with easiness of maintenance of the cells 221 ensured.

Since the outer shell of the secondary chamber 223 is also covered with the shielding member 231, a situation in which the fluorescence produced in the primary chamber 222 and leaking through the secondary chamber 223 acts as noise in the magnetic field measurement can be effectively avoided.

The invention is not limited to the embodiments described above, and a variety of changes and improvements can be made to the embodiments described above. Variations will be described below.

### Variation 1

### Cell

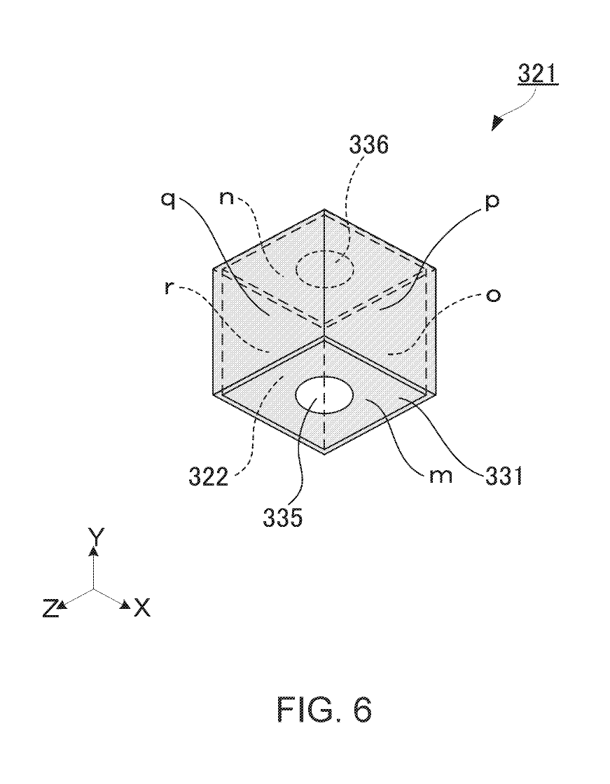

The first embodiment has been described with reference to the configuration in which no shielding member 131 is placed on the surface on which the laser light L is incident (surface a) or the surface through which the laser light L exits (surface b), but the configuration is not necessarily employed. The configuration of a cell according to the present variation will be described with reference to FIG. 6. FIG. 6 is a schematic perspective view showing the cell according to Variation 1. The same configuration portions as those in the first embodiment have the same reference characters, and no redundant description will be made.

A cell 321 shown in FIG. 6 has a primary chamber 322, on which the laser light L is incident, and a shielding member 331. The primary chamber 322 is an internal space surrounded by a roughly cubic outer shell. The laser light L enters the primary chamber 322. The outer shell described above is formed of 6 surfaces, surfaces m, n, o, p, q, and r each having a roughly square shape. The primary chamber 322 encapsulates cesium, and the outer shell maintains airtightness of the cell, as in the first embodiment.

The surface m is the surface on which the laser light L is incident, and the surface n is the surface through which the laser light L having passed through the cell 321 exits. The surfaces o, p, q, and r are surfaces (side surfaces) of the outer shell that are roughly parallel to the Y direction and include surfaces via each of which two cells 321 are adjacent to each other when a plurality cells 321 are arranged to form a cell array 320 (not shown).

The shielding member 331 is provided on part of the outer shell (surfaces m, n, o, p, q, and r), which forms the primary chamber 322, specifically, in the area excluding the area through which the laser light L passes (enters or exits). That is, the shielding member 331 is placed on part of the outer shell described above, specifically, in the area excluding an area 335 of the surface m through which the laser light L passes and an area 336 of the surface n through which the laser light L passes. The areas 335 and 336 each preferably have a size greater than or equal to the beam diameter of the laser light L. The areas 335 and 336, through which the laser light L passes, each have a roughly circular shape, but not necessarily limited thereto. Further, the positions of the areas 335 and 336 do not necessarily roughly coincide with the centers of the surfaces m and n, respectively.

The shielding member 331 can be made of any of the materials described above. The shielding member 331 is a fabric produced by coloring a cotton velvet sheet black by using a coloring agent primarily made of carbon black.

As described above, the cell 321 according to the present variation can provide the following effects in addition to those provided by the first embodiment. Since the shielding member 331 is placed also on part of the surfaces m and n, through which the laser light L passes, specifically, in the area excluding the areas 335 and 336, through which the laser light L passes, the shielding capability of the cell 321 is further improved. As a result, in the cell array 320 (not shown) and the magnetic field measuring apparatus including the plurality of cells 321, optical crosstalk can be further suppressed and detection sensitivity can be further improved.

### Variation 2

### Cell

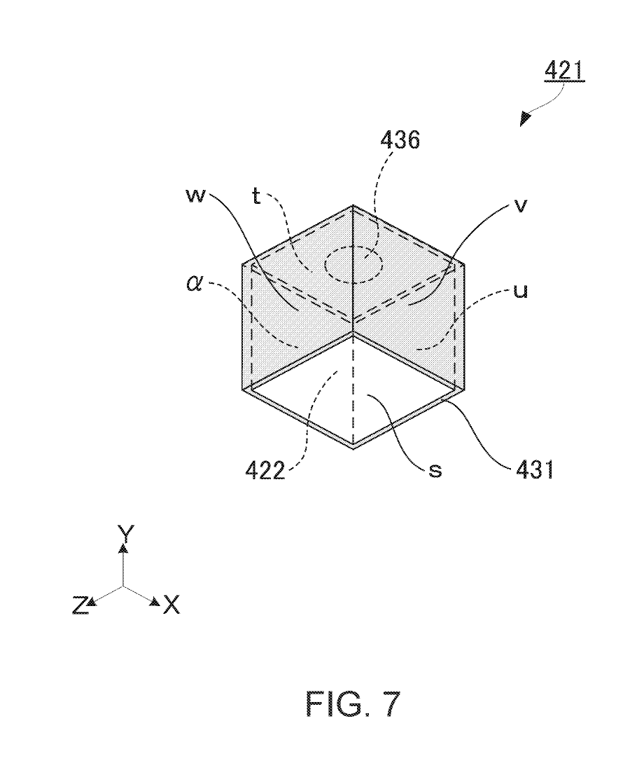

The configuration of a cell according to the present variation will be described with reference to FIG. 7. FIG. 7 is a schematic perspective view showing the cell according variation 2. The same configuration portions as those in the first embodiment have the same reference characters, and no redundant description will be made.

A cell 421 shown in FIG. 7 has a primary chamber 422, on which the laser light L is incident, and a shielding member 431. The primary chamber 422 is an internal space surrounded by a roughly cubic outer shell. The laser light L enters the primary chamber 422. The outer shell described above is formed of 6 surfaces, surfaces s, t, u, v, w, and α each having a roughly square shape. The primary chamber 422 encapsulates cesium, and the outer shell maintains airtightness of the cell, as in the first embodiment.

The surface s is the surface on which the laser light L is incident, and the surface t is the surface through which the laser light L having passed through the cell 421 exits. The surfaces u, v, w, and α are surfaces (side surfaces) of the outer shell that are roughly parallel to the Y direction and include surfaces via each of which two cells 421 are adjacent to each other when a plurality cells 421 are arranged to form a cell array 420 (not shown).

The shielding member 431 is provided on part of the outer shell (surfaces s, t, u, v, w, and α), which forms the primary chamber 422, specifically, on the side surfaces (surfaces u, v, w, and α) and in the area excluding an area 436 of the surface t, through which the laser light L passes. In other words, the surface s or the area 436 is provided with no shielding member 431. That is, Variation 2 differs from the embodiment 1 in that the shielding member 431 is placed also on the surface t, through which the laser light L exits. The area 436 described above preferably has a size greater than or equal to the beam diameter of the laser light L. The shielding member 431 is made of the same material described in the first embodiment.

As described above, the cell 421 according to the present variation can provide the following effects in addition to those provided by the first embodiment. Since the shielding member 431 is placed also on part of the surface t, through which the laser light L exits, specifically, in the area excluding the area 436, through which the laser light L passes, the shielding capability of the cells 421 is further improved. Further, since no shielding member 431 is provided on the surface s, on which the laser light L is incident, the structure of the cell 421 is simplified as compared with the structure in Variation 1 described above, whereby the cell 421 is readily manufactured. As a result, in the cell array 420 (not shown) including the plurality of cells 421 and the magnetic field measuring apparatus, optical crosstalk can be further suppressed in the simplified configuration.

### Variation 3

### Cell

A cell according to the present variation includes a shielding container as the shielding member in place of the fabric or the resin layer described above. That is, a single cell is accommodated in a single shielding container, and a plurality of the shielding containers each including a cell are arranged to form a cell array. To allow the laser light

L to pass through the cells, a corresponding area of each of the shielding containers has an opening. The shielding containers are not necessarily made of a specific material and may be made of any material that is a light absorbent, nonmagnetic material. Specifically, examples of the material may include a rubber material and a resin material with which carbon black or any other substance is kneaded. As a result, after the cell accommodated in a shielding container is exchanged to a new cell, the shielding container is reusable, whereby easiness of maintenance of the cell array and the magnetic field measuring apparatus can be further improved.

The entire disclosure of Japanese Patent Application No. 2016-118613 filed Jun. 15, 2016 is expressly incorporated by reference herein.

What is claimed is:

1. A magnetic field measuring apparatus comprising:
a cell array including a first cell and a second cell that each accommodate a medium that changes a polarization rotation angle of probing light incident on the medium in accordance with an intensity of a magnetic field;
a light source that emits the probing light; and
shielding members with which the first cell and the second cell are provided.

2. The magnetic field measuring apparatus according to claim. 1, wherein the shielding members are provided on adjacent surfaces of the first cell and the second cell.

3. The magnetic field measuring apparatus according to claim 1,
wherein each of the first cell and the second cell has a first chamber on which the probing light is incident, and
the corresponding shielding member is provided on an outer shell that forms the first chamber but only in an area excluding an area through which the probing light passes.

4. The magnetic field measuring apparatus according to claim 1, wherein the shielding members have a light absorbing property.

5. The magnetic field measuring apparatus according to claim 4, wherein the shielding members each include a fabric.

6. The magnetic field measuring apparatus according to claim 4, wherein the shielding members each include a resin layer.

7. The magnetic field measuring apparatus according to claim 1, wherein the medium contains an alkali metal.

8. The magnetic field measuring apparatus according to claim 1, wherein each of the first cell and the second cell accommodates a buffer gas.

9. The magnetic field measuring apparatus according to claim 1, wherein a paraffin film containing aliphatic hydrocarbon having a carbon number of 20 or more is provided on an inner surface of each of the first cell and the second cell.

10. The magnetic field measuring apparatus according to claim 1,
wherein each of the first cell and the second cell has a first chamber on which the probing light is incident and a second chamber that communicates with the first chamber, and
each of the first cell and the second cell is provided with the shielding member.

11. A cell array comprising:
at least a first cell and a second cell each accommodating a medium that changes a polarization plane orientation of probing light incident on the medium in accordance with an intensity of a magnetic field; and
shielding members with which the first cell and the second cell are provided,
wherein the at least first cell and second cell are so disposed as to be adjacent to each other.
